(19) United States
(12) Patent Application Publication
(10) Pub. No.: US 2014/0096779 A1
(43) Pub. Date: Apr. 10, 2014

(54) DEVICE FOR USE WITH POST-OPERATIVE SHOE

(71) Applicant: Cynthia Roggenkamp, Lincoln, NE (US)

(72) Inventor: Cynthia Roggenkamp, Lincoln, NE (US)

(21) Appl. No.: 13/647,764

(22) Filed: Oct. 9, 2012

(51) Int. Cl. A61F 5/37

(52) U.S. Cl. 128/882

(57) ABSTRACT

A device for use with a post-operative shoe to protect the foot of the wearer of such shoe. The device comprises a first member and a second member, wherein the first member is substantially U-shaped and has a first and second terminus, wherein the first terminus and second terminus of the substantially U-shaped first member are of substantially equal length relative to its central axis, and wherein the second member is partially U-shaped and has a first and second terminus, wherein the first terminus of the partially U-shaped second member is substantially shorter than the second terminus of the partially U-shaped second member relative to its central axis, and further wherein the first and second members are attached to each other at their central axis at a generally 90 degree angle, and wherein the first terminus and second terminus of the first member, and the first terminus and second terminus of the second member extend in the same direction, and wherein the first terminus of the second member includes means for receiving a front portion of a post-operative shoe. When the device is secured to a post-operative shoe, the foot of the wearer of such shoe is protected against trauma.

### FIELD OF THE INVENTION

The present invention relates generally to the protection of feet of wearers of post-operative shoes, and more particularly to a device for use with a post-operative shoe to protect the foot of the wearer of such shoe.

### BACKGROUND OF THE INVENTION

Foot problems resulting from poor circulation, open sores, and ulcers are commonplace, particularly among geriatric patients and patients suffering from diabetes. These problems make it painful to walk and, therefore, often require surgery. Forefoot surgeries, including bunionectomies, metatarsophalangeal joint exostectomies, hammertoe operations, claw and mallet toe corrections, and neuroma excisions, are commonly done everyday throughout the world by general orthopedic surgeons, foot and ankle orthopedic surgeons, and podiatrists. Post-operative shoes are commonly used throughout the recovery period from forefoot surgery to both assist in the healing process and protect the surgical site from damage. Achieving an adequate level of comfort with a post-operative shoe is often cumbersome for a patient, and frequently a post-operative shoe that is most comfortable to a patient may offer the least protection from trauma to the foot. A need, therefore, exists for a device that can be used with a post-operative shoe to protect against trauma to the foot inside such shoe.

### SUMMARY OF THE INVENTION

Disclosed herein is a device for use with and attachment to a post-operative shoe to protect the feet and toes of the wearer of such shoe. The device comprises a first member and a second member, wherein the first member is substantially U-shaped and has a first and second terminus, wherein the first terminus and second terminus of the substantially U-shaped first member are of substantially equal length relative to its central axis, and wherein the second member is partially U-shaped and has a first and second terminus, wherein the first terminus of the partially U-shaped second member is substantially shorter than the second terminus of the partially U-shaped second member relative to its central axis, and further wherein the first and second members are attached to each other at their central axis at a generally 90 degree angle, and wherein the first terminus and second terminus of the first member, and the first terminus and second terminus of the second member extend in the same direction, and wherein the first terminus of the second member includes means for receiving a front portion of a post-operative shoe, and thereby permitting the attachment of the device to the post-operative shoe.

### BRIEF DESCRIPTION OF THE DRAWINGS

The device described herein may best be understood by reference to the following drawing, wherein:

FIG. 1 is a representation of a preferred embodiment of the device attached to a post-operative shoe.

### DETAILED DESCRIPTION

The invention disclosed herein will be described with respect to particular embodiments and with reference to certain drawings, but it is to be understood that the invention is limited thereby. The drawings described are only schematic and are non-limiting. In the drawings, the size of some of the elements may be exaggerated or distorted and not drawn on scale for illustrative purposes. Where an indefinite or definite article is used when referring to a singular noun e.g. “a” or “an,” “the,” this includes a plural of that noun unless something else is specifically stated.

Disclosed herein is a device for use with and attachment to a post-operative shoe to protect the feet and toes of the wearer of such shoe. The device comprises a first member and a second member, wherein the first member is substantially U-shaped and has a first and second terminus, wherein the first terminus and second terminus of the substantially U-shaped first member are of substantially equal length relative to its central axis, and wherein the second member is partially U-shaped and has a first and second terminus, wherein the first terminus of the partially U-shaped second member is substantially shorter than the second terminus of the partially U-shaped second member relative to its central axis, and further wherein the first and second members are attached to each other at their central axis at a generally 90 degree angle, and wherein the first terminus and second terminus of the first member, and the first terminus and second terminus of the second member extend in the same direction, and wherein the first terminus of the second member includes means for receiving a front portion of a post-operative shoe, thereby permitting the attachment of the device to the post-operative shoe.

In a preferred embodiment, a first arm is attached to the first terminus of the first member, a second arm is attached to the second terminus of the first member, and a third arm is attached to the second terminus of the second member. Preferably, a space exists between each of the first arm and the first terminus of the first member, second arm and the second terminus of the first member, and third arm and the second terminus of the second member, such space being sufficient to accommodate one or more straps that may be inserted therethrough to secure the device to the post-operative shoe.

The first and second members, and first, second, and third arms, are preferably made of a lightweight material so as to minimize the load on the wearer of a post-operative shoe having the device secured thereon, and yet hard enough to offer protection to the wearer from environmental impacts. Preferred materials include plastic, aluminum, and titanium.

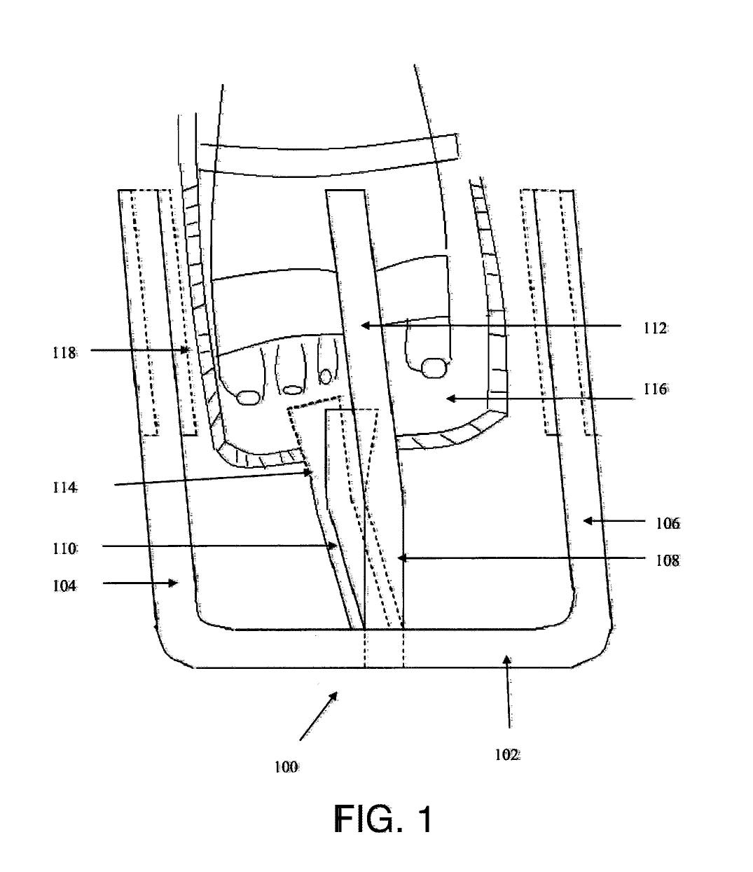

Referring to FIG. 1, the device 100 disclosed herein comprises a first member 102 having a central axis and a first terminus 104 and second terminus 106, and a second member 108 having a central axis and a first terminus 110 and second terminus 112, wherein the first member 102 is attached at its central axis to the second member 108 at its central axis at a generally 90 degree angle. The first member 102 is substantially U-shaped, and the first terminus 104 and second terminus 106 of the first member 102 are of substantially equal length relative to its central axis. The second member 108 is partially U-shaped, and its first terminus 110 is substantially shorter than its second terminus 112 relative to its central axis. The first terminus 110 of the second member 108 includes means 114 for receiving a front portion of a post-operative shoe 116. In a preferred embodiment, a first arm 118 is attached to the first terminus 104 of the first member 102, a second arm 120 is attached to the second terminus 106 of the first member 102, and a third arm (not shown) is attached to the second terminus 112 of the second member 108.

When the device described herein is secured to a post-operative shoe, the foot of the wearer of such shoe is protected from trauma to the foot, thereby permitting proper healing of the foot that led to the use of the post-operative shoe in the first instance.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The Abstract of the disclosure will allow the reader to quickly ascertain the nature of the technical disclosure. It is submitted with the understanding that it will not be used to interpret or limit the scope or meaning of the following claims.

What is claimed is:

1. A device for use with and attachment to a post-operative shoe to protect the feet and toes of the wearer of such shoe, the device comprising:
a first member, wherein the first member is substantially U-shaped and has a first and second terminus, wherein the first terminus and second terminus of the substantially U-shaped first member are of substantially equal length relative to its central axis;
a second member, wherein the second member is partially U-shaped and has a first and second terminus, wherein the first terminus of the partially U-shaped second member is substantially shorter than the second terminus of the partially U-shaped second member relative to its central axis,
wherein the first and second members are attached to each other at their central axis at a generally 90 degree angle, wherein the first terminus and second terminus of the first member, and the first terminus and second terminus of the second member extend in the same direction, and wherein the first terminus of the second member includes means for receiving a front portion of a post-operative shoe.

2. The device of claim 1, further comprising a first arm, second arm, and third arm, wherein the first arm is attached to the first terminus of the first member, the second arm is attached to the second terminus of the first member, and the third arm is attached to the second terminus of the second member.
